(19) United States
(12) Patent Application Publication
(10) Pub. No.: US 2002/0143281 A1
(43) Pub. Date: Oct. 3, 2002

(54) Disposable finger cover

(76) Inventor: Diana Wiley, Detroit, MI (US)

Correspondence Address: DIANA WILEY, 11061 NASHVILLE, DETROIT, MI, 48205, US

(21) Appl. No.: 09/818,225

(22) Filed: Mar. 28, 2001

(51) Int. Cl. A61F 13/00

(52) U.S. Cl. 602/62; 602/60

(57) ABSTRACT

DFC'S are used for two applications. The first application is a finger covering that is made of cotton weaving on the outside and plastic latex on the inside, referred to herein as DFC#1. DFC#1 is used to apply mdeication to open wounds, cuts, sores and can be used anywhere on the body as needed for sanitary protection. The second application is a finger covering that is made of cotton weaving on the outside and cotton weaving on the inside, referred to as DFC#2. The DFC#2 is used for putting on and taking off facial make-up and cosmetics, cleaning dirt out of eyes and ears, and good to use on all the body where sanitation is necessary to ensure non-contamination from fingers and nails. Also, DFC'S guard against spreading contagious bacteria whil applying medication and cosmetics.

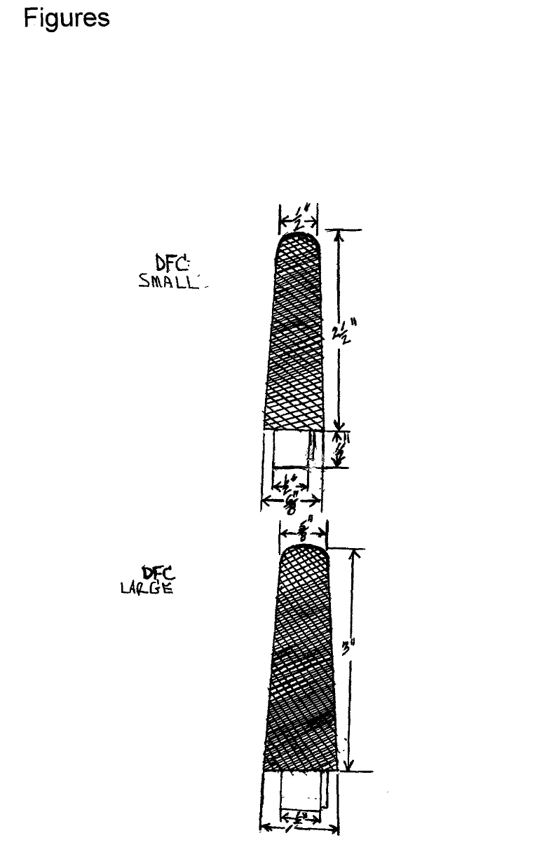

### BRIEF DESCRIPTION OF DRAWINGS

I have attached one page showing two drawings of the disposable finger cover the drawing at the top of the page is the bottom of the page is captioned as DFC large, representing two sizes of the DFC'S. Both DFC small and DFC large comes with two tabs, one on each side, to assist in putting the DFC on the finger. The two types of DFC'S (DFC#1 AND DFC#2) described in the following section will come in the sizes shoun in the drawing.

### DETAILED DESCRIPTION

I would like to obtain a patent for two types of dispoasble finger covers (DFC) are used for two different applications. The first DFC is constructed with a plastic latex lining inside, herein referred to as DFC#1. DFC#1 is used to apply medication to open wounds, cuts, sores and can be used anywhere on the body as needed for sanitary protection.

The second DFC is constructed with a cotton weaving outside covering and a cotton weaving inside lining, herein referred to as DFC#2. DFC#2 is used for putting on and taking off facial make-up and cosmetics, cleaning dirt out of eyes and ears, and is good to use on babies to apply ointments and creams.

Both DFC#1 and DFC#2 can be used daily on al parts of the body where sanitaiton is nessary to ensure non-contanination from fingers and nails. Also, the DFC'S guards against spreading contagious bacteria while applying medication and cosmetics. Both DFC'S will be constructed with a tab in front and a tab in back to assist in putting the DFC'S on the fingers and to take the DFC'S off the fingers. The DFC'S will be m,aintained in individually wrapped paper packets.

DFC#1 and DFC#2 will be available in small and large sizes for female and male fingers. The small DFC will be one-half inch (½″) in width at the top or end of the finger covering and descend down to the bottom of the finger covering to six-eights inch (6/8″) in width. This means that the DFC opening is one inch at the top or end of the finger opening to an inch and one-half at the bottom of the DFC finger opening. The large DFC will be five-eights inches (⅝″) in width at the top or end of the finger covering and descend down to the bottom of the finger covering to one inch (1″) in width. This means that the DFC opening is one & one-quarter inches at the top or end of the finter opening to two inches at the bottom of the DFC finger opening.

1. I claim that the DFC'S can be used daily on all parts of the body where sanitaion is necessary to ensure non-contamination from fingers and nails.

2. I claim that the DFC#1 is used to apply medication to wounds, cuts, sores and can be used anywhere on the body as needed for protection.

3. I claim that the DFC#2 is used for putting on and taking off facial make-up and cosmetics, cleaning dirt out of eyes and ears.

4. I claim that the DFC'S guards against spreading contagious bacteria while applying medication and cosmetics to the body.

5. I claim that the DFC'S are good to use on babies to apply ointments and creams.
